(19) United States
(12) Patent Application Publication
(10) Pub. No.: US 2015/0136150 A1
(43) Pub. Date: May 21, 2015

(54) DURABLE SKIN MARKING COMPOSITIONS

(71) Applicant: OP-MARKS, INC., Bogart, GA (US)

(72) Inventors: Stephen Bruce Lober, Athens, GA (US); Ian Askill, Colorado Springs, CO (US)

(73) Assignee: OP-MARKS, INC., Boart (GA)

(21) Appl. No.: 14/356,069

(22) PCT Filed: Nov. 5, 2012
(86) PCT No.: PCT/US12/63572

§ 371 Date: May 2, 2014

Related U.S. Application Data
(60) Provisional application No. 61/556,169, filed on Nov. 4, 2011.

(51) Int. Cl. A61L 31/08; A61L 31/10; A61L 31/16; A61B 19/00

(52) U.S. Cl. CPC A61L 31/08; A61B 19/54; A61L 31/10; A61L 31/16; A61B 2019/545
USPC 128/898; 106/31.03; 523/105; 106/31.02; 514/785

(57) ABSTRACT

Disclosed herein are methods and compositions of durable skin marking compositions that remains legible and visible on the skin after being treated with an aqueous, alcohol-based solution. The skin marking composition of the present invention includes a cyanoacrylate component, a solvent, and a colorant. The composition can further comprise one or more of viscosity modifiers/thickening agents, stabilizers, plasticizers, formaldehyde scavengers, polymerization accelerators, perfumes, adhesion promoters, and antibacterial, anti-fungal, anti-viral, or anti-microbial agents. This abstract is intended as a scanning tool for purposes of searching in the particular art and is not intended to be limiting of the present invention.

### CROSS-REFERENCE TO RELATED APPLICATIONS

This application claims the benefit of U.S. Application No. 61/556,169 filed Nov. 4, 2011, which is hereby incorporated by reference in its entirety.

### FIELD OF THE INVENTION

The present invention relates generally to durable skin marking compositions, and more specifically to skin marking compositions that remain visible after surgery preparation.

### BACKGROUND OF THE INVENTION

It is common to use marking pens on surgical patients, whether in human or veterinary practice, prior to surgery. Surgeons will make marks to indicate planned lines of incision, to delineate key anatomical landmarks, and to denote correct surgical sites. It is important that these marks remain visible after pre-operative preparation of the skin for surgery to insure surgical precision and to prevent wrong-site surgery.

In the past, surgeons have used ink compositions that contain harmful and deleterious toxins, such as magic markers and commercially-available felt-tip pens. A disadvantage, however, with the aforementioned marking devices and less harmful devices of the prior art is that their markings can be removed by skin preparations containing alcohol. The higher the concentrations of alcohol, the more likely the markings are effectively completely removed from easy sight. Such preoperative skin preparations are already in common use. However, as these preparations have been shown to be more efficacious in reducing the number of antibiotic resistant bacteria within the surgical field when compared to non-alcohol based skin preparations, they are now being used at increasing rates.

The prevalence of alcohol-based skin preparations has led to a recent need for a surgical marking formulation that is biocompatible, remains legible (i.e., does not smear or run), and is visible to a clinical team after a region of skin has been marked and subsequently treated with one of the preoperative preparations.

Cyanoacrylate compositions are well known and widely used as rapidly curing adhesives for various substrates, including human tissue. Particularly, cyanoacrylate compositions have been used as permanent adhesives to repair surgical lacerations to internal organs and blood vessels. Cyanoacrylate compositions have also been used to permanently seal wounds and prevent blood or other bodily fluid leakage from the wounds. Cyanoacrylate compositions have not, however, been used as a durable, yet removable, surgical marking composition. It is to such compositions that the present invention is primarily directed.

### SUMMARY OF THE INVENTION

In accordance with the purpose(s) of the present invention, as embodied and broadly described herein, the invention, in one aspect, relates to durable skin marking compositions that remain legible and visible on the skin. In various aspects, the disclosed skin marking compositions remain legible and visible on the skin even after treatment with an aqueous or alcohol-based solution, but are removable after use. The disclosed durable compositions for skin marking comprise a cyanoacrylate component, a solvent, and a colorant. The disclosed durable skin marking compositions optionally further comprise one or more additives, as further described herein.

In one aspect, the invention relates to a durable skin marking composition comprising a cyanoacrylate component, a solvent, a colorant, and a viscosity modifier. In another aspect, the invention relates to a durable skin marking composition comprising a cyanoacrylate component, a solvent, and a viscosity modifier pre-imbued with a colorant.

In another aspect, the invention relates to a method of marking skin for an activity comprising providing a disclosed skin marking composition, marking skin with the composition prior to the activity, and removing at least a portion of the composition after the activity.

In various aspects, the present invention has applicability to surgical procedures. In one aspect, the present disclosure provides a method of marking skin in preparation of surgical activity. In a further aspect, a disclosed marking composition is used by a clinician, for example, a surgeon, to mark, for example, a planned line of incision, to delineate anatomical landmarks, or to clearly label correct surgical sites. In a still further aspect, the durable skin marking composition provides markings that remain visible after pre-operative preparation of the skin. In a yet further aspect, the durable skin marking composition is then later removed, for example, after completion of the surgical activity.

Additional aspects of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION OF THE INVENTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such can, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Although exemplary aspects of the invention are explained in detail, it is to be understood that other aspects are contemplated. Accordingly, it is not intended that the invention is limited in its scope to the details of construction and arrangement of components set forth in the following description or illustrated in the drawings. The invention is capable of other aspects and of being practiced or carried out in various ways. Also, in describing the exemplary aspects, specific terminology will be resorted to for the sake of clarity.

All publications mentioned herein are incorporated herein by reference to disclose and describe the methods and/or materials in connection with which the publications are cited.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, example methods and materials are now described.

As used in the specification and the appended claims, the singular forms “a,” “an” and “the” include plural referents unless the context clearly dictates otherwise. Thus, for example, reference to “an additive” includes mixtures of two or more additives.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “optional” or “optionally” means that the subsequently described event or circumstance can or cannot occur, and that the description includes instances where said event or circumstance occurs and instances where it does not. For example, the phrase “optional additive” means that the additive can or cannot be present in the compositions.

By “comprising” or “containing” or “including” is meant that at least the named compound, component, material, element, or method step is present in the composition or article or method, but does not exclude the presence of other compounds, components, materials, elements, method steps, even if the other such compounds, components, material, elements, method steps have the same function as what is named.

As used herein, the term “dermatologically acceptable” generally refers to an ink that is substantially permanent or indelible while remaining non-toxic.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific aspect or combination of aspects of the methods of the invention.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included. For example if a particular element or component in a composition or article is said to have 8% weight, it is understood that this percentage is relation to a total compositional percentage of 100%.

Each of the materials disclosed herein are either commercially available and/or the methods for the production thereof are known to those of skill in the art.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### Durable Marking Compositions

As briefly described above, the present disclosure provides, in one aspect, durable compositions for skin marking comprising a cyanoacrylate component, a solvent, and a colorant. In a further aspect, the durable compositions can further comprise, for example, viscosity modifiers, plasticizers, stabilizers, accelerants, formaldehyde scavenging compounds, perfumes, adhesion promoters, anti-bacterial agents, anti-fungal agents, anti-viral agents, anti-microbial agents, or a combination of the foregoing.

In various aspects, the durable marking compositions comprise a cyanoacrylate component. In one aspect, the cyanoacrylate component comprises one or more biocompatible or dermatologically acceptable cyanoacrylates. In this aspect, cyanoacrylate bases are widely used as rapidly curing adhesives for various substrates, including human tissue. In liquid form, cyanoacrylate exists as a monomer. When exposed to free radicals or anions, especially hydroxyl ions present in water, the monomer undergoes an exothermic hydroxylation reaction that results in rapid polymerization.

In a further aspect, the cyanoacrylate preferably comprises a longer chain cyanoacrylate derivative. That is, investigations using cyanoacrylates as tissue adhesives, i.e. suture replacements, have reported that shorter-chain cyanoacrylate derivatives (e.g., 2-methyl-, 2-ethyl-) exhibit greater tissue toxicity than the longer chain derivatives (n-butyl-, 2-octyl-). In a still further aspect, the cyanoacrylate can comprise either FDA approved or unapproved cyanoacrylates. Currently, n-butyl- and 2-octyl-compounds are the only cyanoacrylates approved by the U.S. Food and Drug Administration (“FDA”) for use with skin or other living human tissue in the United States, although 2-ethyl-cyanoacrylate is approved for use outside the United States. In a preferred aspect, the cyanoacrylate comprises n-butyl-cyanoacrylate (BCA), 2-octyl-cyanoacrylate (OCA), 2-ethyl-cyanoacrylate (ECA), or ethoxyethyl cyanoacrylate (EECA), or a combination thereof.

Application of cyanoacrylate to mammalian tissue, whether skin or other living tissue, has focused on development of adhesives for surgical use involving tissue adhesion or wound repair. Prior to the present invention, it was unknown to use cyanoacrylate in a skin marking composition, and suggestion of the same was met with active disagreement. Typical uses include adhesives for repair of surgical lacerations to internal organs and blood vessels, as well as wound sealing to prevent blood or other bodily fluid leakage. These uses commonly constitute in vivo sealants, fillers for internal cavities or voids, demonstrate slow reaction kinetics for use with mucosal type tissues, or have specific biodegradable properties. Typically, these adhesives are clear or imbued with a small amount of color from a limited class of colorants to just minimally achieve a tint. The nature of the monomeric cyanoacrylate component limits the selection of colorants and the amounts thereof that can be added. For example, too much colorant or the wrong class of colorant can cause premature polymerization of the composition, rendering it inoperable for its intended use.

In one aspect, the present marking compositions comprising a solvent can overcome some of the obstacles described above. In a further aspect, the solvent suitable for use in the disclosed compositions are preferably those solvents compatible with the selected colorant, cyanoacrylates, or other component. In a still further aspect, the solvent is preferably biocompatible. In an even further aspect, the solvent is miscible with the selected colorant, cyanoacrylates, or other component. In a yet further aspect, the solvent can preferably be viscosified, is non-toxic, and volatile. In a still further aspect, the solvent can be polar or non-polar, provided the solvent is capable of dissolving the colorant, stable with the cyanoacrylate, compatible with thickening agents, inexpensive and sufficiently volatile. In a yet further aspect, at least a portion of the colorant and the cyanoacrylate are dissolved in the solvent phase. In an even further aspect, the solvent phase can solvate substantially all the dispersed colorant and cyanoacrylate. In a yet further aspect, the solvent will further comprise solvated viscosity modifier. In a still further aspect, the solvent can comprise solvated uncoated colorant, cyanoacrylate, and a polymer agent.

The examples report the results of 36 investigated solvents, from which a preferred solvent can be selected to meet the desired criteria. Naturally, routine adjustments in the composition formulation can be made to employ a desired solvent. In one aspect, the solvent for use in the disclosed marking compositions comprises butanone, 1,4 dioxane, ethyl acetate, t-propyl, tetrahydrofuran (THF), or toluene, or combinations thereof. In a preferred aspect, the solvent comprises tetrahydrofuran (THF).

In a further aspect, the solvent can be present in the composition in any desired amount. In a still further aspect, the solvent is present in an amount of at least 20% by weight, at least 30% by weight, at least 40% by weight, at least 50% by weight, or even at least 60% by weight of the composition. In a yet further aspect, the solvent is present in an amount from at least 10% by weight to about 90% by weight of the composition, for example, 15, 20, 25, 30, 35, 40, 45, or 47.5%, or even 50, 55, 60, 65, 70, 75, 80, or 85% by weight of the composition. In an even further aspect, the solvent can be present in any range between the foregoing values, for example, from at least 20% by weight to about 80% by weight, or from at least 30% to about 70% by weight, or from at least 40% by weight to about 60% by weight, or even from at least 45% to about 55% by weight of the composition.

Previous work with cyanoacrylate adhesives has seen even pigments known to be compatible, such as anthraquinones and hydroxyanthraquinones, limited to less than about 1% by mass of the total composition, and typically ranging from about 0.05 to about 0.2% by mass. At such low levels, the compositions of the prior art do not contain sufficient colorant to impart enough contrast against the skin to be effectively used as a distinctly visible marking agent.

In one aspect, the marking compositions of the present invention, however, comprise colorant greater than about 0.5% by mass of the total composition. In a further aspect, the colorant is present in an amount greater than about 1% by mass of the total composition. In a still further aspect, the colorant is present in an amount greater than about 2.5% by mass of the total composition. In an even further aspect, the colorant can be present in any amount from at least 0.5% to about 15% by weight of the composition, for example, 0.5, 1.0, 1.5, 2.0, 2.5, 5, 7, 8, 9, 10, or 15% by weight of the composition. In a still further aspect, the colorant can be present in any range between the foregoing values, for example from at least 0.5% to about 10% by weight, or from at least 0.5% to about 7.5% by weight, or even from at least 0.5% to about 5% by weight of the composition. In another aspect, the upper limit of colorant carrying capacity can be dependent on the specific colorant, the cyanoacrylate monomer, the viscosity modifier, and the solvents used in the final composition. In a further aspect, the anionic and free radical stabilizing agents typically have less an effect on the colorant carrying capacity.

In one aspect, the present marking compositions can comprise a polymeric agent. In a further aspect, the polymer agent can be used in formulations not otherwise capable to provide enough color contrast to increase the colorant-carrying capacity to a sufficient level in order to achieve contrast against human skin or other external tissue suitable for use as a surgical marking agent. In a still further aspect, the polymer agent comprises a compatible polymeric component imbued with an increased amount of colorant, such as pigments, dyes, or other colored particles (i.e., colored in the ultraviolet, visible, or infrared, fluorescent, radio-opaque, etc.) in comparison to a monomeric cyanoacrylate. In a yet further aspect, the polymeric agent can be used with a deeply colored polymeric component to increase contrast provided by the final formulation by preventing premature polymerization of the cyanoacrylate monomer that would normally be caused by such amounts of the colorant.

In another aspect, depending on the polymeric component employed, the polymeric agent can also be used to increase viscosity to provide the present formulation suitable for use as a non-bleeding marking agent. In a further aspect, the polymeric agent is preferably selected to function as a viscosity modifier as well as to increase the colorant carrying capacity of the final formulation. In a still further aspect, the polymeric agent comprises certain polymeric components that can also act as plasticizers for the cyanoacrylate-based composition, preventing unwanted cracking of the marks produced when applied to flexible tissue, such as skin.

In one aspect, the marking compositions of the present invention, however, comprise polymeric component greater than about 0.5% by mass of the total composition. In a further aspect, the polymeric component is present in an amount greater than about 1% by mass of the total composition. In a still further aspect, the polymeric component is present in an amount greater than about 2.5% by mass of the total composition. In an even further aspect, the polymeric component can be present in any amount from at least 0.5% to about 25% by weight of the composition, for example, 0.5, 1.0, 1.5, 2.0, 2.5, 5, 7, 8, 9, 10, 15, 20, or 25% by weight of the composition. In a still further aspect, the polymeric component can be present in any range between the foregoing values, for example from at least 0.5% to about 20% by weight, or from at least 1.0% to about 15% by weight, or even from at least 1.5% to about 10% by weight of the composition.

In various aspects, the colorant in the present composition can comprise, for example, dyes, pigments, contrast agents, colored particles, fluorescent materials, radio-opaque materials, or a combination of the foregoing. In a further aspect, the colorant can be chemically capped or otherwise coated or encapsulated to prevent premature polymerization caused by the nature of the colorant or to otherwise enhance compatibility with the polymeric base and other constituents of the present formulation. In a still further aspect, the colorant can comprise FDA approved colorants for use with food and food packaging. In a yet further aspect, the colorant selected preferably provides markings clearly perceptible to the unaided eye. In an even further aspect, the colorant preferably provides markings visible under visible light, ultraviolet light, and/or x-ray exposure. In a still further aspect, the colorant preferably provides markings visible to doctors using surgical or diagnostic equipment to perform surgical activities.

In a further aspect, the colorant is cyanoacrylate compatible and biocompatible. In a still further aspect, the colorant is selected from one or more of the following compounds including, but not limited to, [phthalocyaninato (2-)]copper, D&C Violet No. 2 (1-hydroxy-4-[(4-methylphenyl)amino]-9,10-anthracenedione), Solvent Green No. 3 (1,4-di-p-toluidino-9,10-anthraquinone (PTA)), FD&C Yellow No. 6 (disodium 6-hydroxy-5-[(4-sulphonatophenyl)azo]naphthalene-2-sulphonate), FD&C Red No. 3 (9-(o-carboxyphenyl)-6-hydroxy-2,4,5,7-tetraiodo-3H-xanthen-3-one, disodium salt, monohydrate), FD&C Blue No. 2 (2-(13-dihydro-3-oxo-5-sulfo-2H-indol-2-ylidene)-2,3-dihydro-3-oxo-1H-indole-5-sulfonic acid disodium salt), C.I. Solvent Green 5 (3,9-Perylenedicarboxylicacid,bis(2-methylpropyl)ester), D&C Green No. 6 (1-Hydroxy-4-[(4-methylphenyl)amino]-9,10-anthracenedione), C.I. Acid Red 50 (sulforhodamine G), or C.I. Acid Red 52 (sulforhodamine B monosodium salt). In a yet further aspect, the colorant can comprise many other pigments and/or capped or otherwise encapsulated colorants to impart varying degrees of opacity, for example, fluorescence, or radio-opacity.

As briefly described, the disclosed durable marking compositions can comprise one or more additives. In a one aspect, the additive comprises an additive for the cyanoacrylate component. In a further aspect, the additive can include, among others, viscosity modifiers/thickening agents, stabilizers (anionic and/or free radical polymerization inhibitors), plasticizers, formaldehyde scavengers, polymerization accelerators, perfumes, and adhesion promoters, and can be used in exemplary aspects of the present invention. In a further aspect, the marking composition can further comprise one or include one or more adjuvants, for example, anti-bacterial, anti-fungal, anti-viral, or anti-microbial agents, or a combination thereof.

In one aspect, viscosity modifiers/thickening agents can be used in the present marking compositions to increase viscosity of a cyanoacrylate component, thereby controlling deposition. In a further aspect, viscosity modifiers suited for cyanoacrylate include, but are not limited to polymeric agents such as poly-cyanoacrylates (polymeric alpha 2-cyanoacrylates); acrylate resins such as polyalkyl methacrylates, polyalkyl acrylates, and poly(methyl methacrylates); cellulose derivatives such as nitrocellulose, cellulose acetates, and cellulose esters including cellulose acetate butyrate; poly(vinyl alkyl ethers); polycaprolactone, lactic-acid caprolactone, polyglycolic acid, lactic glycolic acid, polylactic acid; and copolymers thereof. In a still further aspect, thixotropic thickening agents such as silica gels (e.g., fumed silica treated with silyl isocyanate) also can be used to modify the viscosity of the cyanoacrylate component. In a yet further aspect, carbon black silica can also be used as a thixotropic thickening agent, simultaneously imparting color to the formulation.

In one aspect, the marking compositions of the present invention comprise viscosity modifiers/thickening agents greater than about 0.5% by weight of the total composition. In a further aspect, the viscosity modifiers/thickening agents are present in an amount greater than about 1% by weight of the total composition. In a still further aspect, the viscosity modifiers/thickening agents are present in an amount greater than about 2.5% by weight of the total composition. In an even further aspect, the viscosity modifiers/thickening agents can be present in any amount from at least 0.5% to about 25% by weight of the composition, for example, 0.5, 1.0, 1.5, 2.0, 2.5, 5, 7, 8, 9, 10, 15, 20, or 25% by weight of the composition. In a still further aspect, the viscosity modifiers/thickening agents can be present in any range between the foregoing values, for example from at least 0.5% to about 20% by weight, or from at least 1.0% to about 15% by weight, or even from at least 1.5% to about 10% by weight of the composition.

In exemplary aspects of the present invention, viscosity modifiers/thickening agents can include nitrocellulose, cellulose acetate, cellulose esters, poly(methyl methacrylate), or polycaprolactone, or combinations thereof. In a further exemplary aspect, the viscosity modifiers/thickening agents do not exceed 25% by weight of the total composition.

In another aspect, the marking compositions can comprise stabilizers, or polymerization inhibitors, or compounds that can be added to extend the shelf-life of the present cyanoacrylate component by protecting against premature polymerization by anionic or free radical exposure. In a further aspect, suitable stabilizers useful for use in the present invention with human skin or other external tissues include, but are not limited to, anionic stabilizers such as sulfur dioxide, nitric oxide, boron triflouride, sulfonic acid, and lactone, and free radical stabilizers, such as hydroquinone, nitrohydroquinone, monomethyl ether hydroquinone, monoethyl ether hydroquinone, and catechol. In a still further aspect, mixtures of the foregoing are acceptable so long as the mixture of stabilizers does not adversely affect the desired polymerization rate and characteristics of the cyanoacrylate monomer.

In one aspect, the marking compositions of the present invention can comprise stabilizers, or polymerization inhibitors in any desired amount. In a further aspect, the stabilizers, or polymerization inhibitors are present in an amount of at least 1 ppm. In a further aspect, the stabilizers, or polymerization inhibitors are present in an amount of at least 100 ppm. In a still further aspect, the stabilizers, or polymerization inhibitors are present in an amount of at least 500 ppm. In an even further aspect, the stabilizers, or polymerization inhibitors can be present in any amount from at least 1 ppm to about 5,000 ppm, for example, 1, 5, 10, 20, 50, 100, 200, 300, 400, 500, 1000, 1,500, 2,000, or 2,500, or 5,000 ppm. In a still further aspect, the stabilizers, or polymerization inhibitors can be present in any range between the foregoing values, for example from at least 1 ppm to about 500, or from at least ppm to about 250 ppm, or from at least 500 ppm to about 5,000 ppm, or even 500 ppm to about 2,500 ppm.

In an exemplary aspect, the anionic stabilizer, when present, can comprise from about 1 to about 500 ppm of the total composition. In a further exemplary aspect, the radical stabilizer, when present, can comprise from about 500 to about 5,000 ppm of the total composition. In an even further aspect, the radical stabilizer can comprise hydroquinone, nitrohydroquinone, monomethyl ether hydroquinone, monoethyl ether hydroquinone, catechol, or a combination of two or more of the foregoing. In a yet further aspect, exemplary biocompatible stabilizers in accordance with the present invention include butylated hydroxyl anisole, sulfur dioxide, or hydroquinone, or a combination thereof. In preferred aspects, the marking composition comprises the minimally effective amount of stabilizing agents needed to achieve acceptable clinical shelf life as a marking agent while not adversely affecting desired polymerization properties, including cure rate.

In another aspect, plasticizers can be used with disclosed cyanoacrylate compositions to impart more flexibility to the cured formulation. In a further aspect, suitable plasticizers for use in the disclosed compositions include, but are not limited to, difunctional aromatic esters, phosphates, phosphonates, and mono- or difunctional alphiatic esters of acids. In a still further aspect, specific examples of plasticizers include, but are not limited to, acetyl tri-n-butyl citrate, acetyl trihexyl citrate, dioctyl phthalate, dibutyl phthalate, dimethyl sebecate, phosphates such as triethyl phosphate and tri(p-cresyl)phosphate, glyceryl triacetate, glyceryl tributyrate, dimethyl sebacate, diethyl sebacate, dioctyl adipate, dioctyl glutarate, butyl stearate, lauric acid, and mixtures thereof. However, phthalates have recently come into question as potentially having biotoxic effects, especially in infants and children. In another aspect, certain polymeric agents can be used in the present marking compositions to produce a plasticizing effect when incorporated into monomeric cyanoacrylate. In a further aspect, examples of suitable polymeric agents include, but are not limited to, polyethylene glycol esters, polyester gluterates, or polyester adiapates, or combinations thereof.

In one aspect, the marking compositions of the present invention can comprise plasticizers in any desired amount. In a further aspect, plasticizers can comprise at least about 0.5% by weight of the total composition. In a further aspect, the plasticizers are present in an amount greater than about 1% by weight of the total composition. In a still further aspect, the plasticizers are present in an amount greater than about 2.5% by weight of the total composition. In an even further aspect, the plasticizers can be present in any amount from at least 0.5% to about 25% by weight of the composition, for example, 0.5, 1.0, 1.5, 2.0, 2.5, 5, 7, 8, 9, 10, 15, 20, or 25% by weight of the composition. In a still further aspect, the plasticizers can be present in any range between the foregoing values, for example from at least 0.5% to about 20% by weight, or from at least 1.0% to about 15% by weight, or even from at least 1.5% to about 10% by weight of the composition.

In a further aspect, exemplary biocompatible plasticizers for the present invention are tributyl citrate, acetyl tributyl citrate, or butyl sterate, or a combination of the foregoing. In a further exemplary aspect, the plasticizers are present in amounts that do not exceed 20% by weight of the total composition.

Formaldehyde is a well-known byproduct of cyanoacrylate degradation and is the primary compound leading to tissue toxicity. Because the marking compositions of the present invention are intended for external surgical marking of skin, nails, etc., this toxicity risk is less of a concern than for internally used cyanoacrylate adhesives. However, since the surgical incision may cut through a given marking comprising a disclosed marking composition, thereby exposing internal living tissue to the ink, and all aspects of biocompatibility should be considered. Therefore, the present invention can comprise formaldehyde scavenging agents to reduce formaldehyde concentration levels as the ink begins to degrade. In a further aspect, the formaldehyde scavenging agents can be in free or microencapsulated form. In a still further aspect, the formaldehyde scavenging agent can comprise any formaldehyde scavenging compounds compatible for use with a cyanoacrylate component. In a yet further aspect, examples of suitable formaldehyde scavenging agents include, but are not limited to, alcohols, proteins, mercaptans, amines, amides, imides, sulfites, bisulfites, nitriles, cyclic ketones, and combinations thereof.

In one aspect, the marking compositions of the present invention can comprise formaldehyde scavenging agents in any desired amount. In a further aspect, formaldehyde scavenging agents can comprise at least about 0.5% by weight of the total composition. In a further aspect, the formaldehyde scavenging agents are present in an amount greater than about 1% by weight of the total composition. In a still further aspect, the formaldehyde scavenging agent are present in an amount greater than about 2.5% by weight of the total composition. In an even further aspect, the formaldehyde scavenging agent can be present in any amount from at least 0.5% to about 25% by weight of the composition, for example, 0.5, 1.0, 1.5, 2.0, 2.5, 5, 7, 8, 9, 10, 15, 20, or 25% by weight of the composition. Ina still further aspect, the formaldehyde scavenging agent can be present in any range between the foregoing values, for example from at least 0.5% to about 20% by weight, or from at least 1.0% to about 15% by weight, or even from at least 1.5% to about 10% by weight of the composition.

In an exemplary aspect, biocompatible formaldehyde scavenging compounds for the present invention include sodium bisulfite and/or urea, or combinations thereof.

In another aspect, polymerization accelerators, or initiators, can be incorporated into the present composition. In one aspect, polymerization accelerators or initiators can be used in circumstances where the addition of other polymeric substances or solvents are needed to achieve beneficial contrast for use as a marking composition, resulting in prolonged curing time beyond clinically acceptable parameters. In a further aspect, polymerization accelerators or initiators can be used if the addition of other materials to enhance shelf-life or utility (i.e., stabilizers, scavengers, additional viscosity or plasticizing agents, or initiators) prolongs curing time beyond clinically acceptable parameters.

In one aspect, the marking compositions of the present invention can comprise polymerization accelerators or initiators in any desired amount. In a further aspect, polymerization accelerators or initiators can comprise at least about 0.5% by weight of the total composition. In a further aspect, the polymerization accelerators or initiators are present in an amount greater than about 1% by weight of the total composition. In a still further aspect, the polymerization accelerators or initiators are present in an amount greater than about 2.5% by weight of the total composition. In an even further aspect, the polymerization accelerators or initiators can be present in any amount from at least 0.5% to about 25% by weight of the composition, for example, 0.5, 1.0, 1.5, 2.0, 2.5, 5, 7, 8, 9, 10, 15, 20, or 25% by weight of the composition. In a still further aspect, the polymerization accelerators or initiators can be present in any range between the foregoing values, for example from at least 0.5% to about 20% by weight, or from at least 1.0% to about 15% by weight, or even from at least 1.5% to about 10% by weight of the composition.

In an exemplary aspect, polymerization accelerators or initiators suitable for use in the disclosed cyanoacrylate systems include, but are not limited to, hydroxyls, water, polyalkylene oxides, crown ethers, carboxylate, sulfur compounds such as thiols, molecules containing amino, imine, imide, or amide groups, sodium phosphates, and many metallo-organic compounds.

In another aspect, the marking compositions can comprise a surfactant. In a further aspect, the surfactant can be incorporated into the cyanoacrylate component directly to enhance dispersion of the initiator within the solution. In a still further aspect, an initiator can also be added via the delivery system for the marking composition. In a yet further aspect, the initiator can be contained within a porous tip or coated along the internal, solution-contacting sides of applicator tip to enhance cure upon delivery to skin. In an even further aspect, the initiator can be added extraneously as a spray or wipe to skin prior to marking with a disclosed marking composition or applied as a spray after marking skin to quicken cure time.

In one aspect, the marking compositions of the present invention can comprise surfactants in any desired amount. In a further aspect, surfactants can comprise at least about 0.5% by weight of the total composition. In a further aspect, the surfactants are present in an amount greater than about 1% by weight of the total composition. In a still further aspect, the surfactants are present in an amount greater than about 2.5% by weight of the total composition. In an even further aspect, the surfactants can be present in any amount from at least 0.5% to about 25% by weight of the composition, for example, 0.5, 1.0, 1.5, 2.0, 2.5, 5, 7, 8, 9, 10, 15, 20, or 25% by weight of the composition. In a still further aspect, the surfactants can be present in any range between the foregoing values, for example from at least 0.5% to about 20% by weight, or from at least 1.0% to about 15% by weight, or even from at least 1.5% to about 10% by weight of the composition

In another aspect, the disclosed marking composition can be sterilized by, for example, chemical, physical, and/or irradiation methods.

In exemplary aspects, compositions according to the present invention act at room temperature, approximately 20° C., when applied to skin, nails, or other external body part. In further exemplary aspect, the compositions also comprise elements that are biocompatible. Additionally, the process by which the compositions dry and polymerize via exothermic reaction is biocompatible.

In various aspects, the marking compositions of the present invention can be applied by any suitable applicator or method of application. In one aspect, the applicator or method of application enables the user to control the deposition and thereby achieve effective use as a marking medium. In a further aspect, the applicator or method of application comprises a finger, brush, sponge, spray, stylus, stencil, stamp, sticker, marker, or pen, or combination thereof.

In one aspect, the marking composition is applied using a marker pen. In a further aspect, the marker pen can dispense the composition via a felt tip, fiber tip, brush tip, sponge tip, other porous tip, ball tip, gel dispensing tip, or other tips used to apply ink from reservoir barrels in marker pens. In a still further aspect, the marker pen can be scaled to a variety of shapes and sizes so long as the marking pen is capable of providing a desired mark on the tissue of a surgical patient. Because the spread of infectious disease is of concern, the marking pen can be, in another aspect, unit dosed and disposed of after a single use. Accordingly, one or more surgical marking pens can be packaged in a manner that is capable of providing an indication to a prospective user as to whether a marking pen has been previously used.

In another aspect, the marker pen can comprise a reservoir that has an opening for delivering a suitable amount of the present marking composition from the reservoir to the nib. In one aspect, the nib is a conventional felted foam tip capable of receiving the composition stored in the reservoir and shaped and sized such that when the nib is drawn across the tissue of a surgical patient, the skin marking composition can be deposited onto the surface of the patient's tissue and leave a desired mark. In another aspect, the nib itself is the marking agent reservoir. In a further aspect, a conventional felt tip nib is preloaded with a suitable amount of the skin marking composition such that the marking agent is operable only until the composition loaded into the nib is either deposited onto a desired surface or dries up after being exposed to air.

In yet another aspect, a marker pen can comprise a sufficient amount of the present composition for a single use. In a further aspect, the marker pen can be sized and shaped to form a friction fit within a cap member sized and shaped to cover the marking nib to prevent the composition from drying out and rendering the marking pen inoperable.

As described above, the disclosed skin marking compositions are designed to remain legible and visible on the skin even after treatment with an aqueous or alcohol-based solution, but are nevertheless removable. In one aspect, the present marking compositions comprising cyanoacrylate can be removed by several solvents including acetone, methyl-ethyl-ketone, nitromethane, and gamma-butyrolactone. In a further aspect, acetone and gamma-butyrolactone are preferred for removal because they exhibit the least toxicity and sensitivity to humans. In a further aspect, marks left by the present marking composition can be removed by application and rubbing with a solution containing acetone or gamma-butyrolactone, whether via pre-moistened and packaged wipes or direct application of the solution via a sponge applicator or cotton ball or the like.

### Articles

In various aspects, the disclosed durable marking compositions can be used in producing articles, for example, a marking article. The durable marking compositions itself can be also made into a marking article, for example, a marking sticker or applique. In a further aspect, the durable marking compositions can form components of marking articles or systems. Marking articles include, for example, brush, sponge, spray, stylus, stencil, stamp, sticker, a tattoo, applique, marker, or pen. In an exemplary aspect, the disclosed marking compositions are used in a marker pen. In a further aspect, the marker pen can dispense the composition via a felt tip, fiber tip, brush tip, sponge tip, other porous tip, ball tip, gel dispensing tip, or other tips used to apply ink from reservoir barrels in marker pens. In a still further aspect, the marker pen can be scaled to a variety of shapes and sizes so long as the marking pen is capable of providing a desired mark on the tissue of a surgical patient. In a yet further aspect, the marking pen is unit dosed and disposed of after a single use.

### Methods of Manufacturing Durable Marking Compositions

In various aspects, the durable skin marking composition of the present invention can be manufactured by various methods. The disclosed compositions can be prepared by a variety of methods involving admixing the described materials with any additional additives in the formulation. In a further aspect, the durable marking compositions of the present invention can be by prepared by any suitable mixing means known in the art.

In one aspect, the invention relates to methods of preparing a durable marking composition comprising: admixing a cyanoacrylate component, a colorant, and a solvent.

In another aspect, the invention relates to methods of preparing a durable marking composition comprising: admixing a cyanoacrylate component, a colorant, a solvent, and a viscosity modifier. In a still further aspect, the composition prepared according to the present invention can comprise any desired combination of components described herein.

In another aspect, the invention relates to methods of preparing a durable marking composition comprising: admixing a cyanoacrylate component comprising n-butyl-cyanoacrylate, 2-ethyl-cyanoacrylate, 2-octyl-cyanoacrylate, or ethoxyethyl cyanoacrylate, or a combination of two or more of the foregoing, a colorant, a solvent, and a viscosity modifier.

In a further aspect, the method of preparing further comprises mixing in a stabilizer, a plasticizer, a formaldehyde scavenging compound, a polymerization accelerator, an agent comprising an anti-bacterial agent, anti-fungal agent, anti-viral agent, antimicrobial agent, or a combination of two of more of the foregoing.

### Methods of Marking

As described herein, various aspects of the present invention provide a durable skin marking composition that are useful for marking tissue, for example, mammalian skin tissue. Advantageously, the disclosed marking compositions remain legible and visible on the skin after being treated with an alcohol-based solution that is dermatologically acceptable. As such, the properties render the present formulation suitable for use as a durable marking agent for skin or other mammalian tissue. In one aspect, the durable compositions are suitable for marking tissue, for example, the tissue of a patient. In a further aspect, the tissue can comprise any tissue biologically compatible with the present marking compositions. In a still further aspect, the tissue comprises mammalian tissue, such as, skin or nails.

Thus, in one aspect, the invention provides a method of marking skin for an activity comprising providing a disclosed skin marking composition comprising a cyanoacrylate component, a solvent, and a colorant, marking skin with the composition prior to the activity, and removing at least a portion of the composition after the activity. In a further aspect, the activity can comprise and clinical activity. In still further aspect, the activity can therapeutic or diagnostic. In a yet further aspect, the activity can be invasive or non-invasive. In a preferred aspect the activity is surgical activity.

In another aspect, the present invention provides a method of marking skin in preparation of surgical activity comprising providing a skin marking composition comprising a cyanoacrylate component, a solvent, and a colorant, marking skin with the composition prior to the surgical activity to denote correct surgical sites prior to the activity, wherein the marking remains visible after pre-operative preparation of the skin for surgery, and removing at least a portion of the composition after surgery. In a further aspect, the marking can comprise one or more lines or areas. In a further aspect, the marking can be used to delineate key anatomical landmarks, or planned lines of incision. In a still further aspect, the composition is removed by rubbing with a solvent selected from the group consisting of acetone and gamma-butyrolactone.

In various aspects, the cyanoacrylate component imparts durability to the disclosed marking composition. Cyanoacrylates are particularly well suited for the present invention because of their ability to rapidly polymerize at room temperature without the use of an added catalyst when applied to a substrate, and their ability to adhere well to tissue, such as mammalian skin. Once cured, cyanoacrylate is durable to exposure to water, blood, other bodily fluids, common solvents such as alcohols, and newer pre-surgical skin preparation solutions containing alcohols, iodine, and/or chlorhexadine. Thus, in one aspect, the disclosed marking compositions comprising a cyanoacrylate component provide a protective barrier in which the colorant is contained. In this aspect, the durable marking compositions can limit premature removal of the marking upon exposure to such bodily fluids and/or skin preparation solutions. In a further aspect, the durable marking compositions can fully prevent its premature removal upon exposure to bodily fluids and/or skin preparation solutions.

In various aspects, the present invention also includes at least the following aspects:

In one exemplary aspect, is a method of marking skin for an activity comprising providing a skin marking composition comprising a cyanoacrylate component, a solvent, and a colorant, marking skin with the composition prior to the activity, and removing at least a portion of the composition after the activity

In another exemplary aspect, is the preceding aspect wherein the activity is surgical activity.

In another exemplary aspect, is a method of marking skin in preparation of surgical activity comprising providing a skin marking composition comprising a cyanoacrylate component, a solvent, and a colorant, marking skin with the composition prior to the surgical activity to denote correct surgical sites prior to the activity, wherein the marking remains visible after pre-operative preparation of the skin for surgery, and removing at least a portion of the composition after surgery.

In another exemplary aspect, is the preceding aspect, wherein the composition is removed by rubbing with a solvent selected from the group consisting of acetone and gamma-butyrolactone.

In another exemplary aspect, is a durable composition for skin marking of the present invention preferably comprises a cyanoacrylate component comprising n-butyl-cyanoacrylate, 2-ethyl-cyanoacrylate, 2-octyl-cyanoacrylate, or ethoxyethyl cyanoacrylate, or a combination of two or more of the foregoing, a colorant, a solvent, and a viscosity modifier, wherein the viscosity modifier of this aspect preferably comprises polycyanoacrylates, acrylate resins, cellulose derivatives, poly(vinyl alkyl ethers), poly caprolactone, lactic acid, caprolactone, polyglycolic acid, lactic glycolic acid, polylactic acid, and thixotropic thickening agents.

In another exemplary aspect, is a durable composition for skin marking of the preceding aspect can further comprise a stabilizer, a plasticizer, a formaldehyde scavenging compound, a polymerization accelerator, an agent comprising an anti-bacterial agent, anti-fungal agent, anti-viral agent, antimicrobial agent, or a combination of two of more of the foregoing, and an adhesion promoter.

In another exemplary aspect, is a durable composition for skin marking of the present invention preferably comprises a cyanoacrylate component comprising n-butyl-cyanoacrylate, 2-ethyl-cyanoacrylate, 2-octyl-cyanoacrylate, or a combination of the foregoing, a solvent, and a viscosity modifier, wherein the viscosity modifier is pre-imbued with a colorant.

In another exemplary aspect, is a durable composition for skin marking of the present invention preferably comprises a cyanoacrylate component, a solvent, and a colorant comprising dyes, pigments, inks, or a combination of the foregoing, wherein the colorant is preferably greater than about 0.5% by mass of the total composition.

In another exemplary aspect, is a durable composition for skin marking of the present invention preferably comprises a cyanoacrylate component, a solvent, and a colorant comprising fluorescent particles, radio-opaque particles, or a combination of the foregoing, wherein the colorant is preferably greater than about 0.5% by mass of the total composition.

Without further elaboration, it is believed that one skilled in the art can, using the description herein, utilize the present invention. The following examples are included to provide addition guidance to those skilled in the art of practicing the claimed invention. The examples provided are merely representative of the work and contribute to the teaching of the present invention. Accordingly, these examples are not intended to limit the invention in any manner.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### EXAMPLES

The following examples are put forth so as to provide those of ordinary skill in the art with a complete disclosure and description of how the methods, devices, and systems disclosed and claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

1. Selection of Solvents for Durable Marker Ink

Potential solvents were evaluated for compatibility with dye and cyanoacrylates, ability to be viscosified, toxicity and volatility. The initial 36 solvents identified as potential solvents, along with their boiling point (BP), vapor pressure (VP), volatility (VL), and toxicity (TOX) are described in Table 1 below. For all the examples herein, boiling point is given in ° C. Vapor pressure is provided as mmHg at the indicated temperature, given in ° C. Volatility values are relative volatility compared to butyl acetate. Toxicity is provided as LD₅₀ oral dose administered to rat in mg/kg.

TABLE 1

| Solvent | BP | VP | VL | TOX |
|---|---|---|---|---|
| Acetone | 56 | 400@39.5 | 7.7 | 2000 |
| Butanone | 80 | 400@60 | 2.7 | 2737 |
| Butoxyethanol | 171 | 0.8@20 | 0.07 | 470 |
| Butyl acetate | 126 | 15@25 | 1 | 10768 |
| t-Butyl acetate | 118 | 17.8@25 | 1.5 | 13400 |
| Chloroform | 62 | 160@20 | NA | 908 |
| Cyclohexane | 81 | 95@20 | 2.6 | 12705 |
| Cyclohexanone | 156 | 5@25 | 0.29 | 1000 |
| Diacetone alcohol | 172 | 0.95@20 | 0.14 | 4000 |
| Dichloropropane | 97 | 40@20 | −1 | 1947 |
| Diethylene glycol | 244 | 1@92 | NA | NA |
| Diethyl ether | 35 | 440@20 | 37.5 | 1215 |
| Diisobutyl ketone | 168 | 1.7@20 | 0.2 | 5750 |
| Diisopropyl ketone | 124 | 13.3@37 | 0.17 | 3536 |
| 1,4, Dioxane | 101 | 27@20 | 2.7 | 4200 |
| Ethyl acetate | 77 | 76@20 | 6 | 5620 |
| Ethoxyethyl acetate | 156 | 2@20 | −1 | 2700 |
| 2-ethyl hexanol | 183 | 0.36@20 | 0.01 | 3730 |
| Heptane | 98 | 40@20 | 4.3 | 3200 |
| Hexane | 69 | 132@20 | 8.3 | 28710 |
| Isoprene | 34 | 560@20 | 28.6 | 2100 |
| Methoxy ethanol | 124 | 6.2@20 | −1 | 2370 |
| 2-methoxy ethyl ether | 162 | 3@25 | NA | NA |
| Methoxy propanol | 146 | NA | 0.3 | 8532 |
| Methyl cyclohexane | 101 | 37@20 | 2.99 | 2250 |
| Methyl cyclohexanone | 165 | 3.3@20 | 0.29 | 2140 |
| Methylene chloride | NA | NA | NA | NA |
| Methyl isobutyl ketone | 116 | 16@20 | 1.6 | 1600 |
| Methylisopropylketone | 94 | 42@20 | 2.9 | 3078 |
| Nitromethane | 102 | 28@20 | 1.39 | 940 |
| PGPE (Arcosolv) | 150 | 1.7@20 | 0.21 | 2504 |
| Propyl acetate | 102 | 25@20 | 3.0 | 6640 |
| t-propyl acetate | 89 | 59@25 | 5 | 6750 |
| THF | 66 | 129@20 | 8 | 1650 |
| Toluene | 111 | 22@20 | 2.24 | 636 |
| Xylene | 138 | 9@20 | 0.7 | 3910 |

During the initial evaluation process, any solvent that was designated as carcinogenic or poisonous, or had a volatility of less than 2.2 relative to butyl acetate were not assessed as a potential solvent for this investigation. After further review, methyl cyclohexane and propyl acetate were also not selected in this evaluation as likely having very similar solvating profiles relative to cyclohexane and isopropyl acetate, respectively. The remaining 13 solvents described in Table 2 were then subjected to further analysis. In addition to the parameters described in Table 1, the 13 solvents were also assigned a relative ranking for volatility (RV) and a relative ranking for toxicity (RT). Further, customary cost per liter of solvent (CC) was included in the analysis and assigned a relative cost ranking (RC). Next, a Cumulative Preference Rank (CPR) was determined as the product of the relative rankings for volatility, toxicity, and cost. The data and rankings for the 13 solvents are provided in Table 2 below.

TABLE 2

| Solvent | BP | VP | VL | RV | TOX | RT | CC | RC | VTC | CPR |
|---|---|---|---|---|---|---|---|---|---|---|
| Acetone | 56 | 400@39.5 | 7.7 | 4 | 200 | 4 | 14.38 | 1 | 16 | 1 |
| Butanone | 80 | 400@60 | 2.7 | 11 | 2737 | 3 | 18.45 | 2 | 66 | 9 |
| Cyclohexane | 81 | 95@20 | 2.6 | 11 | 12705 | 1 | 17.68 | 2 | 22 | 5 |
| Diethyl ether | 35 | 440@20 | 37.5 | 1 | 1215 | 5 | 29.90 | 4 | 20 | 4 |
| 1,4 Dioxane | 101 | 27@20 | 2.7 | 11 | 4200 | 3 | 37.65 | 5 | 165 | 12 |
| Ethyl acetate | 77 | 76@20 | 6 | 5 | 5620 | 2 | 23.93 | 3 | 30 | 6 |
| Heptane | 98 | 40@20 | 4.3 | 7 | 3200 | 3 | 30.40 | 5 | 105 | 11 |
| Hexane | 69 | 132@20 | 8.3 | 4 | 28710 | 1 | 25.80 | 4 | 16 | 1 |
| Isoprene | 34 | 560@20 | 28.6 | 1 | 2100 | 4 | 29.06 | 4 | 16 | 1 |
| 3-Methyl | 94 | 42@20 | 2.9 | 10 | 3078 | 9 | 91.60 | 9 | 810 | 13 |
| 2-butanone | | | | | | | | | | |
| t-propyl | 89 | 59@25 | 5 | 6 | 6750 | 2 | 20.68 | 3 | 36 | 7 |
| acetate | | | | | | | | | | |
| THF | 66 | 129@20 | 8 | 4 | 1650 | 4 | 22.63 | 3 | 48 | 8 |
| Toluene | 111 | 22@20 | 2.24 | 15 | 636 | 6 | 12.98 | 1 | 90 | 10 |

Next, the solvents were tested for miscibility and compatibility with cyanoacrylate esters. Briefly, beginning with 3 ml samples each of butyl cyanoacrylate (BCA), octyl cyanoacrylate (OCA) and ethoxyethyl cyanoacrylate (EECA), 3 ml of each solvent was added drop-wise to each cyanoacrylate sample and agitated to aid mixing. Observations of the samples were taken immediately and then at regular intervals over 7 days. Miscibility and compatibility of the samples were assessed using duration of time before exhibiting an increase in viscosity. As a result of the foregoing analysis, three solvents, acetone, isoprene and 3-methyl 2-butanone, were removed from the solvent candidates due to apparent reactions with the cyanoacrylate esters. The remaining 10 solvents were ranked according to their compatibility with the cyanoacrylate esters based primarily on their compatibility with BCA over 7 days, secondarily on their compatibility with OCA, and finally on their miscibility with EECA. The miscibility and compatibility data and rankings for the 13 solvents are provided in Table 3 below.

TABLE 3

| Solvent | | | | | | | | | | | |
|---|---|---|---|---|---|---|---|---|---|---|---|
| BCA | BCA | BCA | BCA | OCA | OCA | OCA | OCA | EECA | EECA | EECA | EECA |
| Time | | | | | | | | | | | |
| 0 | 1 Day | 2 Day | 7 Day | 0 | 1 Day | 2 Day | 7 Day | 0 | 1 Day | 2 Day | 7 Day |

| | | | | | | | | | | | | |
|---|---|---|---|---|---|---|---|---|---|---|---|---|
| Acetone | OK | OK | V | VV | OK | OK | OK | OK | OK | OK | OK | OK |
| Butanone | OK | OK | OK | OK | OK | V | VV | VV | OK | OK | OK | OK |
| Cyclohexane | OK | OK | OK | OK | OK | V | VV | VV | 2Lyr | 2Lyr | 2Lyr | 2Lyr |
| Diethyl ether | OK | OK | OK | OK | OK | OK | OK | OK | OK | OK | OK | OK |
| 1,4 Dioxane | OK | OK | OK | OK | OK | OK | OK | VVV | OK | OK | OK | OK |
| Ethyl acetate | OK | OK | OK | OK | OK | OK | V? | VV | OK | OK | OK | OK |
| Heptane | OK | PlY | LtY | LtY | OK | OK | VV | VV | 2Lyr | 2Lyr | 2Lyr | 2Lyr |
| Hexane | OK | OK | OK | OK | OK | OK | OK | OK | 2Lyr | 2Lyr | 2Lyr | 2Lyr |
| Isoprene | RX | NA | NA | NA | NA | NA | NA | NA | NA | NA | NA | NA |
| 3-Methyl | OK | Gry | Blk | Blk | OK | VV | VV | VV | OK | LtBrn | LtGry | LtGry |
| 2-butanone | | | | | | | | | | | | |
| t-propyl | OK | OK | OK | OK | OK | OK | OK | VV | OK | OK | OK | OK |
| acetate | | | | | | | | | | | | |
| THF | OK | OK | OK | OK | OK | VV | VV | VV | OK | OK | OK | OK |
| Toluene | OK | OK | OK | OK | OK | OK | OK | OK | OK | OK | OK | OK |

V = slightly viscous;
VV = viscous;
VVV = very viscous;
Lyr = layers;
Pl = pale;
Lt = light;
Y = yellow;
RX = reacts;
Gry = grey;
Brn = brown;
Blk = black;

The data from Table 3 indicates that the OCA is significantly more sensitive to solvents than the BCA. Without wishing to be bound by a particular theory, the higher sensitivity may be due to the levels of inhibitors in the two CA formulations and can be adjusted if OCA is a preferable additive. The results also suggest that EECA has a different solubility profile than either BCA or OCA. Three of the solvents are not miscible with EECA, as indicated by the multiple layers formed. In one aspect, BCA appears to be the most suitable CA from the data above.

The compatibility of the remaining solvents with polymers used to increase viscosity was evaluated. Initially, compatibility of the solvents was only going to be evaluated with polymethyl methacrylate (MMA). MMA is the most common agent used to increase CA viscosity and has an established history of use in medical implants and cements. However, initial investigation revealed that MMA was not readily soluble in certain preferred solvents. The investigation was expanded to identify other polymers suitable for use to increase viscosity of the solvents. Thus, the initial three polymers tested were MMA, cellulose acetate butyrate (CAB), and acrylonitrile butadiene styrene (ABS). Again, the preliminary results revealed that CAB had a similar solubility profile to MMA. No solvent was able to completely dissolve ABS, although some solvents dissolved a component of ABS leaving a milky solution. For the solvents that were unable to dissolve MMA or CAB, the range of polymers investigated was expanded to include vinyl acetate coethylene (VacoE), polycaprolactone (Cap), polyvinyl ethyl ether (VEE), polyisobutylene (IB), and cellulose triacetate (Cel Ac)

For this test, the polymers were added at 5% by weight to the solvent under test, agitated vigorously for 30 minutes and then placed under gentle rolling for up to 3 days to determine if they would dissolve within this time period. Observations were recorded over the three days of agitation. When the solvent was observed to dissolve the polymer, the supernatant of resultant solution was removed and the viscosity was measured. Cyclohexane and heptane were observed to partially dissolve polyisobutylene, but nevertheless increased the viscosity noticeably. The solvent performance and viscosity data are shown in Table 4 below. Due to the apparently limited options for viscosifying diethyl ether and heptane, they were removed from further consideration for this investigation.

TABLE 4

| Solvent | MMA | CAB | ABS | VAcoE | Cap | VEE | IB | Cel Ac |
|---|---|---|---|---|---|---|---|---|
| Acetone | 2.4 | 1.8 | N | n.d. | n.d. | n.d. | n.d. | n.d. |
| Butanone | 4.5 | 1.8 | N | n.d. | n.d. | n.d. | n.d. | n.d. |
| Cyclohexane | N | N | N | N | N | 4.5 | 59 | N |
| Diethyl ether | N | N | N | N | N | 1.2 | N | N |
| 1,4 Dioxane | 17 | 6.9 | N | n.d. | n.d. | n.d. | n.d. | n.d. |
| Ethyl acetate | 4.7 | 2.3 | N | n.d. | n.d. | n.d. | n.d. | n.d. |
| Heptane | N | N | N | N | N | 1.5 | 11 | N |
| Hexane | N | N | N | N | N | 0.9 | 7.7 | N |
| t-propyl acetate | N | 3.1 | N | n.d. | n.d. | n.d. | n.d. | n.d. |
| THF | 5.3 | 3.1 | n.d. | n.d. | n.d. | n.d. | n.d. | n.d. |
| Toluene | N | N | N | N | 3.7 | 3.1 | 20 | N |

n.d. = not determined;

For the remaining solvents, colorant solubility was assessed in terms of the solubility of D&C Violet #2. Briefly, 10 g of each of the solvents listed in Table 5 was added to 0.4 g of dye in 12 ml test tubes. The samples were shaken for 30 minutes then allowed to stand for 30 minutes. After standing, 5 ml of the top layer was removed and weighed into pre-weighed aluminum weigh boats where the solvent was allowed to evaporate before re-weighing.

Based on these data, two solvents, hexane and cyclohexane, had insufficient dye solubility to be suitable solvents for the present investigation.

TABLE 5

| | Wt. 5 ml | | % dye | PPM dye |
|---|---|---|---|---|
| Solvent | with dye | Wt. dye | soluble | soluble |
| Butanone | 3.9586 | 0.0150 | 0.38 | 3800 |
| Cyclohexane | 3.7802 | 0.0023 | 0.06 | 600 |
| 1,4 Dioxane | 5.0759 | 0.0585 | 1.15 | 11,500 |
| Ethyl acetate | 4.4582 | 0.0152 | 0.34 | 3400 |
| Hexane | 3.2487 | 0.0017 | 0.05 | 500 |
| t-propyl acetate | 4.3172 | 0.0096 | 0.22 | 2200 |
| THF | 4.3275 | 0.0889 | 2.05 | 20,500 |
| Toluene | 4.2858 | 0.0463 | 1.08 | 11,000 |

The top six solvents, based on the relative criteria set forth above, for use in the durable marker ink composition are summarized in Table 6. Also provided in Table 6, are the polymers dissolved (Poly DIS), the resultant viscosity (VISC), cyanoacrylate compatibility (CAC), and ppm dye solubility (PPM), and the comparative ranking based on the investigation criteria.

TABLE 6

| Solvent | BP | VL | TOX | CC | Poly DIS | VISC | CAC | PPM | RANK |
|---|---|---|---|---|---|---|---|---|---|
| Butanone | 80 | 2.7 | 2737 | 18.45 | MMA, CAB | 4.2, 1.8 | O0, E7 | 3800 | 4 |
| 1,4 Dioxane | 101 | 2.7 | 4200 | 37.65 | MMA, CAB | 17.0, 6.9 | O1, E7 | 11,500 | 2 |
| Ethyl acetate | 77 | 6 | 5620 | 23.93 | MMA, CAB | 4.7, 2.3 | O1, E7 | 3400 | 5 |
| t-propyl | 89 | 5 | 6750 | 20.68 | CAB | 3.1 | O2, E7 | 2200 | 6 |
| acetate | | | | | | | | | |
| THF | 66 | 8 | 1650 | 22.63 | MMA, CAB | 5.3, 3.1 | O0, E7 | 20,500 | 1 |
| Toluene | 111 | 2.24 | 636 | 12.98 | CAP, VEE, | 3.7, 3.1, | O7, E7 | 11,000 | 3 |
| | | | | | IB | 20 | | | |

2. Exemplary Durable Skin Marking Composition Formulations

An exemplary durable skin marking composition of the present invention can be prepared as follows, wherein the exemplary amounts represent a relative weight percent: 47.5% tetrahydrofuran; 47.5% butyl cyanoacrylate; 4% polymethyl methacrylate; and stabilizers for the cyanoacrylate. The stabilizers in the composition were as follows: 300 ppm hydroquinone, 1000 ppm butylated hydroxyl, anisole, and 200 ppm sulfur dioxide. The dye (D&C Violet #2, prepared by GluStitch Canada) was present in the composition in a range from 0.5-1%. To the extent the components of an exemplary composition as described do not add to 100% one skilled in the art can appreciate that these reflect relative proportions.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A method of marking skin for an activity comprising:
(a) providing a skin marking composition comprising a cyanoacrylate component, a solvent, and a colorant;
(b) marking skin with the composition prior to the activity; and
(c) removing at least a portion of the composition after the activity.

2. The method of claim 1, wherein the activity is surgery.

3. The method of claim 1, wherein the solvent is tetrahydrofuran.

4. The method of claim 1, wherein the activity is surgery, and wherein
(b) further comprises marking skin with the composition prior to a surgical activity to denote correct surgical sites prior to the activity; wherein the marking remains visible after pre-operative preparation of the skin for surgery; and wherein
(c) further comprises removing at least a portion of the composition after surgery.

5. The method of claim 4, wherein the marking comprises at least one of at least one planned line of incision or key anatomical landmarks.

6. (canceled)

7. The method of claim 4, wherein removing at least a portion of the composition comprises application and rubbing with a solvent selected from the group consisting of acetone and gamma-butyrolactone.

8. (canceled)

9. A durable composition for skin marking, the durable composition comprising
(a) a cyanoacrylate component;
(b) a solvent; and
(c) a colorant.

10. The durable composition for skin marking of claim 9, wherein the cyanoacrylate component is selected from the group consisting of n-butyl-cyanoacrylate, 2-ethyl-cyanoacrylate, and 2-octyl-cyanoacrylate.

11. The durable composition for skin marking of claim 9, wherein the solvent is tetrahydrofuran.

12. The durable composition for skin marking of claim 9, wherein the colorant is selected from the group consisting of anthraquinones, hydroxyanthraquinones, C.I., Solvent Green 5, C.I. Acid Red 50, and C.I. Acid Red 52[phthalocyaninato (2)]copper.

13. (canceled)

14. The durable composition for skin marking of claim 9, further comprising a viscosity modifier.

15. The durable composition for skin marking of claim 14, wherein the viscosity modifier is selected from the group consisting of polycyanoacrylates, acrylate resins, cellulose derivatives, poly(vinyl alkyl ethers), polycaprolactone, lactic acid, caprolactone, polyglycolic acid, lactic glycolic acid, polylactic acid, and thixotropic thickening agents.

16. (canceled)

17. The durable composition for skin marking of claim 9, further comprising an anionic stabilizer in an amount of about 1-500 ppm of the durable composition for skin marking, the anionic stabilizer selected from the group consisting of sulfur dioxide, nitric oxide, boron triflouride, sulfonic acid, and lactone.

18. The durable composition for skin marking of claim 9, further comprising a radical stabilizer in an amount of about 500-5,000 ppm of the durable composition for skin marking, the radical stabilizer selected from the group consisting of hydroquinone, nitrohydroquinone, monomethyl ether hydroquinone, monoethyl ether hydroquinone, and catechol.

19. The durable composition for skin marking of claim 9, further comprising a plasticizer selected from the group consisting of acetyl tri-n-butyl citrate, acetyl trihexyl citrate, dioctyl phthalate, dibutyl phthalate, dimethyl sebecate, phosphates, glyceryl triacetate, glyceryl tributyrate, dimethyl sebecate, diethyl sebacate, dioctyl adipate, dioctyl glutarate, butyl stearate, and lauric acid.

20. (canceled)

21. The durable composition for skin marking of claim 9, further comprising a formaldehyde scavenging compound selected from the group consisting of alcohols, proteins, mercaptans, amines, amides, imides, sulfites, bisulfites, nitriles, and cyclic ketones.

22. The durable composition for skin marking of claim 9, further comprising a polymerization accelerator selected from the group consisting of hydroxyls, water, polyalkylene oxides, crown ethers, carboxylate, sulfur compounds, amino groups, imine groups, imide groups, amide groups, sodium phosphates, and metallo-organic compounds.

23. The durable composition for skin marking of claim 9, further comprising an agent selected from the group consisting of an anti-bacterial agent, anti-fungal agent, anti-viral agent, and anti-microbial agent.

24. The durable composition for skin marking of claim 9, further comprising a perfume.

25. The durable composition for skin marking of claim 9, further comprising an adhesion promoter.

26-38. (canceled)
